(19) United States
(12) Patent Application Publication
(10) Pub. No.: US 2002/0009497 A1
(43) Pub. Date: Jan. 24, 2002

(54) Method of treatment for dermatological disorders and compositions therefor

(76) Inventors: Raymond E. Tobey, Radnor, PA (US); Sharon F. Levy, Philadelphia, PA (US)

Correspondence Address: AVENTIS PHARMACEUTICALS, INC., PATENTS DEPARTMENT, ROUTE 202-206, P.O. BOX 6800, BRIDGEWATER, NJ, 08807-0800, US

(21) Appl. No.: 09/741,585

(22) Filed: Dec. 20, 2000

Related U.S. Application Data
(63) Continuation of application No. PCT/US99/14354, filed on Jun. 24, 1999.
(60) Provisional application No. 60/090,892, filed on Jun. 26, 1998.

(51) Int. Cl. A61K 9/16; A61K 9/50

(52) U.S. Cl. 424/497

(57) ABSTRACT

A composition and method of treatment of dermatological disorders using 5-fluorouracil at levels below about 1.0%. The disorders to be treated include actinic keratosis, non-malignant lesions of the skin and psoriasis.

### CROSS REFERENCE TO RELATED APPLICATIONS

This application is a continuation of PCT/US99/14354, filed Jun. 24, 1999, which claims priority from U.S. Provisional Patent Application No. 60/090,892, filed Jun. 26, 1998.

### FIELD OF THE INVENTION

The present invention relates to a novel method of treatment of dermatological disorders such as actinic or solar keratoses using low levels of 5-Fluorouracil and compositions therefor.

### BACKGROUND OF THE INVENTION

Actinic keratosis is a type of epithelial precancerous lesion. Sun exposure for many years and poor pigmentation of the skin (i.e., light-colored skin) predispose one to developing actinic keratosis. Actinic keratosis has been treated in various ways, including cryosurgery, ionizing radiation in the form of X-rays, and topical chemotherapy such as that using fluorouracil or trichloroacetic acid. Actinic keratosis is sometimes referred to as solar keratosis or senile keratosis. Actinic keratosis is considered by some as a form of carcinoma in situ; actinic keratosis may progress to overt squamous cell carcinoma.

Fluorouracil is clinically effective in treating actinic or solar keratoses and superficial basal cell carcinomas. Fluorouracil, 5-fluoro-2,4(1H,3H)-pyrimidinedione, is an anti-neoplastic antimetabolite. It has been administered systemically or topically. It is believed to operate on pre-cancerous and cancerous cells by interfering with the synthesis of deoxyribonucleic acid (DNA) and to a lesser extent inhibiting the formation of ribonucleic acid (RNA). Evidence shows that when fluorouracil is metabolized in the anabolic pathway, the methylation reaction of deoxyuridylic acid to thymidylic acid is blocked. Fluorouracil is believed in this manner to create a thymine deficiency which causes unbalanced growth and death of the cell. The effects of DNA and RNA deprivation are most marked on those cells which grow more rapidly and take up fluorouracil at a more rapid rate.

Fluorouracil has been used with success to treat actinic keratosis. Various therapy regimes have been recommended. For example, one commercially available product, Efudex®, a 2% or 5% topical solution or a 5% topical cream of 5-fluorouracil supplied by Roche Laboratories Inc., is recommended to be applied twice daily in an amount sufficient to cover the lesions. The usual duration of therapy with Efudex® is estimated by Roche to be from two to four weeks. Another fluorouracil containing product that is commercially available is Fluoroplex®, a 1% topical cream or solution sold by Allergan, Inc. Fluoroplex® is recommended to be applied twice daily over a period of two to six weeks.

More frequent applications of fluorouracil have also been pursued. For example, one study reported effective clearing of the majority of actinic keratosis lesions by applying a 5% fluorouracil cream or solution four times daily for a period of time varying between seven and twenty one days. Unis N. E., “Short-Term Intensive 5-Fluorouracil Treatment Of Actinic Keratoses”, Dermatol Surg, volume 21, number 2, pp. 162-163, February 1995. Pulse dosing has also been used. For example, in one study, ten patients applied a 5% topical 5-fluorouracil composition one to two days per week for an average of 6-7 weeks. The method reported an average clearing of 98% of the lesions. Pearlman D L, “Weekly Pulse Dosing: Effective and Comfortable Topical 5-Fluorouracil Treatment of Multiple Facial Actinic Keratoses,” J. Am. Acad. Dermatol., Volume 25, number 4, pp. 665-667, October 1991.

These previous therapies have the disadvantage of sometimes causing strong irritation that can cause severe disruptions in a patient's personal or business life. These previous therapies also involved a fairly high concentrations of fluorouracil and/or long duration of treatment. Besides causing severe skin irritation, fluorouracil has certain toxicities. For example, adverse reactions to fluorouracil that occur locally include burning, crusting, allergic contact dermatitis, erosions, erythema, hyperpigmentation, irritation, pain, photosensitivity, pruritus, scarring, rash, soreness and ulceration. Other, non-local, adverse reactions have also been reported, including insomnia, irritability, stomatitis, thrombocytopenia, conjunctival reaction, corneal reaction, nasal irritation and lacrimation.

### SUMMARY OF THE INVENTION

Applicants made the surprising and unexpected discovery that dermatological disorders such as actinic keratoses can be effectively treated with fluorouracil at a lower concentration than had been previously thought. Applicants discovered that lower concentrations, such as those below 2% fluorouracil, can be effective even for a shorter duration, with a lower frequency of application than had been previously thought. In particular, Applicants found surprising that a dosage regime employing a 0.5% fluorouracil composition effectively treated actinic keratosis. More particularly, Applicants found that a once a day dosage of 0.5% fluorouracil composition over seven or fourteen days effectively and safely treated actinic keratosis.

### DETAILED DESCRIPTION OF THE INVENTION

The present invention provides a novel therapeutic method and composition involving the use of low dosage levels of fluorouracil, which are designed to avoid or minimize the undesirable side effects from treatment with fluorouracil while still providing the therapeutic advantages.

The method involves topical administration of a composition comprising less than 2% fluorouracil. The composition may be administered from about one to about four times a day, preferably from about one to about two times a day or once a day. The fluorouracil may be used at dosages from about 1.0% to about 0.5 % or less, preferably from less than about 1.0%, and more preferably from about 0.5% or less. The therapy may be administered over a period of about one to eight weeks, preferably over about one to four weeks, and still more preferably over about one to two weeks. In a preferred embodiment, this composition is administered with a frequency of once a day. The percentage of fluorouracil is measured on a weight/weight ratio to the weight of the composition as a whole.

Fluorouracil may be administered in various ways, including topically in the form of a gel, solution, ointment or cream, most preferably in a cream. Fluorouracil also may be administered via injection. Topical compositions may be applied using a variety of different vehicles, such as pads, adhesive strips, and patches. The various compositions of fluorouracil preferably contain pharmaceutically acceptable excipients and formulation aids. It is known in the art how to prepare compositions of fluorouracil of various strengths.

In formulating the topical compositions, any conventional non-toxic, dermatologically acceptable base or carrier in which fluorouracil is stable can be utilized. The preferred compositions for use in this invention are the conventional cosmetic compositions which can contain materials such as one or more of the following: sunscreens (preferably melanins), penetration enhancers, moisturizers, surfactants, emollients, colorants, conditioners, antimicrobials, astringents, detergents, bulking agents such as cellulose, polymers (particularly those sold under the Carbopol® trademark by the B.F. Goodrich Company), etc.

In a preferred embodiment, the fluorouracil composition contains a delivery vehicle comprised of a polymeric bead having a network of pores. A suitable polymeric bead is that described in U.S. Pat. No. 4,690,825. Preferably, the polymeric bead has a network of pores that is substantially noncollapsible upon removal of the active ingredient. Preferably, the polymeric bead is copolymerized from a comonomer pair selected from the groups consisting of vinyl stearate and divinylbenzine and methylmethacrylate and ethylene glycol dimethylmethacrylate. In a preferred embodiment, the polymeric bead has a diameter of about 10 microns to about 100 microns.

The instant invention can be used to treat a variety of dermatological disorders. For example, the invention may be used to treat actinic damage of all sorts, non-malignant lesions of the skin including various forms of warts, further including plantar warts and venereal warts, and psoriasis.

It is envisioned that the invention may be used in combination with additional therapy to provide additional efficacy or to make the fluorouracil treatment better tolerated. For example, the therapy of the invention may be combined with administration of an active ingredient, which is defined as any material that increases the tolerability of fluorouracil therapy or that provides a therapeutic benefit to a dermatological disorder. For example, an active ingredient may be a corticosteroid or a retinoid. When used in combination therapy, the fluorouracil may be used at dosages from about 5.0% to about 0.5 % or less, preferably from less than about 1.0%, and more preferably from about 0.5% or less. The combination therapy may be administered over a period of about eight weeks or less, preferably over about four weeks or less, and still more preferably over about two weeks or less. It is envisioned that the combination therapy may involve application of one or more active ingredients from about one to about four times a day, preferably from about one to about two times a day. Each active ingredient may be administered separately from the other active ingredients or the active ingredients may be administered together in one composition. The combination therapy may be administered through use of a kit containing one or more compositions containing one or more active ingredients.

The following examples will serve to further typify the nature of the invention but should not be construed as a limitation on the scope thereof.

Dose Ranging Studies

### EXAMPLE 1

The objective of this experiment was to investigate the clinical safety and efficacy of three formulations of fluorouracil formulated with a polymeric bead delivery vehicle (hereinafter, these compositions are called “5-FU”): 0.5%, 2.5% and 5.0%, against a vehicle control not containing any 5-fluorouracil (“Vehicle”) and a 5% fluorouracil cream composition not formulated with a polymeric bead delivery vehicle (“5-FU cream”) for the treatment of actinic keratosis. Patients in this vehicle-controlled, evaluator-blinded, parallel group study were randomly assigned to four weeks of twice daily treatment with one of the aforementioned compositions. The duration of treatment was up to 28 days, as tolerated by the patient. The various compositions were applied to the entire face (and anterior bald scalp).

Efficacy was measured by the reduction in actinic keratosis (“AK”) lesion counts from baseline to the post-treatment follow up and by the Physician Global Assessment of Improvement (10 point scale, Grade-4, Much Worse to Grade 5, Cured or 100% improvement)at the post-treatment follow up visit. Also measured was the change from baseline in the Overall Severity of Actinic Keratosis (4 point scale, Grade 0, None to Grade 3, Severe). Efficacy was assessed at the end of a 4 week period that followed the completion of the treatment phase.

Tolerability was assessed by various measures of physician rated facial irritation and patient rated treatment tolerability. Measures of facial irritation (‘irritation index’) included ratings of erythema (redness), edema (swelling), dryness, and erosions (each assessed on a 0, None, to 3, Severe scale). Patient rated tolerability (assessing interference with daily activities and sleep, as well as pain/discomfort and irritation) was ranked on a visual analog scale. Irritation and tolerability were assessed throughout the treatment phase (up to 4 weeks) and throughout the 4 week follow-up phase of the study.

Patient tolerability was also assessed by the incidence of adverse events (side effects, such as irritation), particularly those that necessitated early discontinuation of study treatment applications.

Results

Efficacy

Each of the experimental 5-fluorouracil/polymeric bead compositions was significantly more effective than the composition comprising the polymeric bead alone, that is without fluorouracil. The 0.5% 5-FU demonstrated efficacy at least as good as that of the higher concentration products; and in some instances was numerically (though not Statistically) superior to the 5% products. Actinic keratosis mean percent reductions from baseline were 92% for 0.5% 5-FU, 95% for 2.5% 5-FU, 86% for 5.0% 5-FU, 89% for 5-FU cream and 27% for Vehicle. The proportions of patients cured (100% improved) were 67% for 0.5% 5-FU, 71% for 2.5% 5-FU, 50% for 5.0% 5-FU, 48% for 5-FU cream, and 0% for Vehicle. Global improvement treatment mean scores were 4.4 for 0.5% 5-FU, 4.5 for 2.5% 5-FU, 4.2 for 5.0% 5-FU, 4.2 for 5-FU cream, and 1.2 for Vehicle. The overall severity of actinic keratosis was reduced from baseline treatment mean scores as follows: 1.3 for 0.5% 5-FU, 1.2 for 2.5% 5-FU, 1.0 for 5.0% 5-FU, 1.9 for 5-FU cream, and 0.3 for Vehicle. Proportions of patients with actinic keratosis severity score reductions exceeding one score unit were 54% in the 0.5% 5-FU treatment, 38% in the 2.5% 5-FU treatment, 29% in the 5.0% 5-FU treatment, 24% in the 5-FU cream treatment, and 0% in the vehicle treatment.

Tolerability

At the end of the treatment phase, the severity scores for edema and erosion, as well as the composite irritation index were less severe in the 0.5% and 2.5% 5-FU treatments compared to the 5% 5-cream and the 5-FU cream As evaluated by the patient tolerance measures, 0.5% and 2.5% were consistently better tolerated than 5% 5-FU bead cream. In some instances (e.g., skin irritation) the 0.5% product was even less irritating than the 2.5% product. Among active 5-FU bead treatment groups, the 0.5% 5-FU group had the lowest proportion of patients (32% vs 52% and 96% in the 2.5% and 5% groups, respectively) who discontinued treatment early due to irritation adverse events.

In conclusion, efficacy with the 0.5% 5-FU product was at least as good as the efficacy seen with higher concentration products. The tolerability of the 0.5% product was at the same time better. Fewer patients in the 0.5% group had to stop treatments early due to side effects (primarily facial irritation) than in the higher concentration groups. The irritation index on treatment was lower in the 0.5% (and 2.5%) groups than in the 5% groups. Additionally, use of the lower concentration product has the potential to minimize systemic exposure to the cytotoxic active ingredient, fluorouracil, which has a well-defined toxicity profile.

### EXAMPLE 2

The objective of this experiment was to investigate the safety and efficacy of a 0.5% formulation of fluorouracil in a composition comprising a polymeric bead having a network of pores that are substantially non-collapsible upon removal of the active ingredient (5-FU) as compared to a 5% fluorouracil cream not containing a polymeric bead as mentioned above (5-Cream) for the treatment of actinic keratosis. The study was designed to evaluate differing treatment regimens (different treatment durations and different daily frequencies). Patients in this placebo-controlled, evaluator-blinded, parallel group study were randomly assigned to treatment groups of 5-Cream for two weeks twice daily (5-Cream_{—}2×2), 0.5% 5-FU for two weeks twice daily (5-FU2×2), 0.5% 5-FU for one week once daily (5-FU 1×1), 0.5% 5-FU for one week twice daily(5-FU 1×2), or a vehicle cream comprising a polymeric bead having a network of pores in a substantially non-collapsible form for two weeks, twice a day (Vehicle 2×2). Overall duration of treatment was seven or fourteen days. Efficacy was measured by a reduction in actinic keratosis (AK) lesion counts, the Physician Global Assessment of Improvement, which included a cure rate (as discussed in Example 1), the Overall Severity of Actinic Keratosis (per Example 1) and the Assessment of Actinic Damage (a 7 Grade Categorical scale, 1=Dyspigmentation, 7=Telangectasia).Tolerability was measured by physician irritation ratings, patient tolerability ratings and incidences of adverse events as in Example 1.

Efficacy Results

Actinic keratosis (“AK”) percent reductions from baseline were as follows:

Treatment median scores: 5-Cream 2×2 92%, 5-FU 2×2 100%, 5-FU 1×2 83%, 5-FU 1×1 80%; Vehicle 2×2 11%. Each 5-FU treatment had significantly greater AK percent reduction compared to vehicle.

Each 5-FU treatment had significantly greater Physician Assessment of Global Improvement compared to Vehicle. The treatment mean scores and percent cured (100% improved) were as follows: 5-Cream 2×2 4.2 (44%); 5-FU 2×2 4.1 (53%); 5-FU 1×2 3.5 (28%); 5-FU 1×1 3.4 (19%); and vehicle 2×2 0.2 (11%).

Overall Severity of Actinic Keratosis change from baseline: Each 5-FU treatment had significantly greater overall severity reductions from baseline compared to vehicle. The 5-FU 2×2 and 5-FU 1×2 were not significantly different from the 5-Cream 2×2. The 5-FU 1×1 had a smaller reduction in overall severity compared to 5-Cream 2×2.

Assessment of Actinic Damage change from baseline: Each 5-FU treatment had significantly greater actinic damage reduction from baseline compared to vehicle.

Tolerability

Physician Irritation Index.

Some components of the Physician Irritation Index were more severe in the 5FU_{—}2×2 treatment compared to vehicle. However, the one week treatments had similar irritation index scores as the treatment with the vehicle. Each of the 5-FU treatments had significantly more severe treatment phase dryness compared to 5-Cream. The 5FU_{—}2×2 treatment also had a more severe irritation index score compared to 5-Cream. Some components of the irritation index, of the treatment or post-treatment phase, were less severe in the 5-FU one week treatments compared to 5-Cream.

Patient Treatment Tolerance. Scores were highly variable and did not indicate significant treatment group differences in the global contrasts. The highest treatment mean scores were 2.3 and 2.4 for the treatment phase average AM and PM skin irritation in the 5FU_{—}2×2 treatment. Treatment means for each other treatment and component were less than 2.0 on a scale of 0 to 10.

Safety (General)

None of the active treatment groups had significantly higher incidences of total adverse events or other specific adverse events compared to vehicle.

Frequency Comparisons

Surprisingly, the one week treatment groups (i.e., 5-FU 1×1 and 5-FU 1×2) in which the product was administered either once a day or administered twice a day did not differ in terms of their treatment efficacy or in measures of safety and tolerability. This led to the observation that the 0.5% 5-FU could be applied once a day without compromising desired efficacy or safety.

We claim:

1. A method of treatment of a dermatological disorder comprising administering a composition comprising a pharmaceutically effective amount for treating said disorder of less than 1% fluorouracil and a polymeric bead having a network of pores.

2. The method of claim 1, wherein the dermatological disorder is actinic keratosis.

3. The method of claim 2, wherein the composition comprises about 0.5% fluorouracil.

4. The method of claim 3, wherein the duration of treatment is between about one to about four weeks.

5. The method of claim 4, wherein the duration of treatment is between about one to about two weeks.

6. The method of claim 4, wherein the composition is administered about twice daily or less frequently.

7. The method of claim 6, wherein the composition is administered once daily for about one to about two weeks.

8. The method of claim 6, wherein the composition is administered twice daily for about one week.

9. The method of claim 1, wherein the dermatological disorder is selected from the group consisting of actinic keratosis, non-malignant lesions of the skin, and psoriasis.

10. The method of claim 1 wherein said network of pores is substantially non-collapsible upon removal of the active ingredient.

11. The method of claim 1 wherein said polymeric bead is prepared by copolymerizing a comonomer pair selected from the group consisting of (a) vinyl stearate and divinylbenzene and (b) methyl methacrylate and ethylene glycol dimethylmethacrylate.

12. The method of claim 1 wherein said polymeric bead has a diameter of about ten to about 100 microns.

13. A pharmaceutical composition for treating a dermatological disorder comprising a pharmaceutically effective amount for treating said disorders of less than 1% fluorouracil and a polymeric bead having a network of pores.

14. The composition of claim 13 wherein said network of pores is substantially non-collapsible upon removal of the active ingredient.

15. The composition of claim 13 wherein said polymeric bead is prepared by copolymerizing a comonomer pair selected from the group consisting of (a) vinyl stearate and divinylbenzene and (b) methyl methacrylate and ethylene glycol dimethylmethacrylate.

16. The composition of claim 13 wherein said polymeric bead has a diameter of about ten to about 100 microns.
